(19) United States
(12) Patent Application Publication
(10) Pub. No.: US 2017/0014262 A1
(43) Pub. Date: Jan. 19, 2017

(54) Intraoral Orthosis Device and Method for Manufacturing

(71) Applicant: Thermal Fit, LLC, Cedar City, UT (US)

(72) Inventor: Phillip Dietz, Saint George, UT (US)

(73) Assignee: THERMAL FIT, LLC, Cedar City, UT (US)

(21) Appl. No.: 14/799,109

(22) Filed: Jul. 14, 2015

(51) Int. Cl. A61F 5/56; B29C 43/02

(52) U.S. Cl. CPC A61F 5/566; B29C 43/021; B29K 2105/253

(57) ABSTRACT

An intraoral orthosis device that has a bite tray nested in an outer tray. The bite tray has a higher deformation temperature than the outer tray at an elevated temperature. The thickness of the outer tray's base varies to create a vertex and angle that is similar to the temporomandibular joint. The intraoral orthosis device has two bite trays, a maxillary bite tray and a mandibular bite tray. The bite trays are connected by arms, releasable fasteners, or a combination of the two. Accordingly, another aspect of the present invention is to provide a method to manufacture an intraoral orthosis device without the need of utilizing the skills of a technician or dentist.

### BACKGROUND OF THE INVENTION

Field of the Invention

This invention relates to a device and method for manufacturing the device which prevents interference with normal breathing during sleeping, and more particularly alleviates snoring and sleep apnea.

Prior Art

Sleep apnea occurs when the muscles and tissues in the back of a person's throat relax and collapses the person's airway during sleep. As air attempts to pass through this collapsed airway, tissues vibrate and causes snoring. Sometimes this collapse prohibits airflow so severely that a person may briefly stop breathing. The person's body may automatically responds and wakes the person up, allowing airflow to resume. This pattern of air deprivation and breath resumptions is known as sleep apnea. Sleep apnea can cause multiple complications, including chronic fatigue, high blood pressure, depression, heart attack and more.

The United States of America, Food and Drug Administration (FDA) has approved two different devices to eliminate or reduce sleep apnea. This first device is a continuous positive airway pressure most commonly known as a CPAP. A CPAP devices opens a blocked airway by forcing air down a person's throat through a mask to keep the airway open and eliminating or reducing sleep apnea. There are several limitations to the CPAP device. Many people find a CPAP extremely uncomfortable and loud. Many users stop using the CPAP device.

The second device is an oral appliance device or a month piece. Traditionally, the intraoral orthosis devices comprised of a maxillary bite tray and a mandibular bite tray. The maxillary bite tray and the mandibular bite tray are connected by either a rod or a releasable fastening surface. The intraoral orthosis devices opens a blocked airway by sustaining the bottom jaw forward while ones sleep, maintaining the airway open, and allowing air to flow freely.

There are several limitations of the existing intraoral orthosis devices to control sleep apnea. The temporomandibular joint is a hinge joint that connects the lower jaw to the temporal bones of the skull. As the temporomandibular joint opens, the distance between the crowns of the back of the maxillary and mandibular teeth becomes increasing smaller with respects to the distance between the crowns of the front of the maxillary and mandibular teeth thus creating an angle.

Determining the angle of the temporomandibular joint is essential in constructing intraoral orthosis devices. Due to the complexity of getting the proper temporomandibular angle, manufactures build up acrylic on the crown of the back molar of the mandibular bite tray. When the patient bites down with the maxillary and mandibular bite trays inserted over the teeth, the force of biting down is placed 100% on the back molar where the buildup of acrylic was placed. This causes sore muscles, sore back molars and other problems. Many times the user may simply stop using the intraoral orthosis devices.

Yet another limitation is the placement of locking devices. Due to the technician not being able to get the proper temporomandibular angle, locking devices that connect the mandibular bite tray and the maxillary bite tray are connected on the anterior or interior of the teeth causing irritation and pain to the gums or tongue.

Yet another limitation is the material and hardware utilized to manufacture an intraoral orthosis devices. To connect the rod or any other locking device to the bite tray, technicians generally install anchors in the bite trays. The technician place an anchor on the anterior of the back molar of the mandibular bite tray and on the anterior of the canine of the maxillary bite tray by applying acrylic and acrylic liquid around the anchors, teeth and gums. With the maxillary and mandibular bite trays placed in a normal biting position, the mandibular and maxillary bite trays are connected together by a rod screwed into the anchors. If the length between the anchor placed on the back molar of the mandibular and the anchor placed on the canine of the maxillary is longer or shorter than the rod, the technician has to reconstruct the bite trays and replace the anchors with acrylic. Also, if the acrylic on the mandibular or maxillary bite trays protrude from the bite trays in a fashion that does not allow the arm to connect the mandibular bite tray and the maxillary bite tray, the technician has to either shave down the acrylic to allow the arm to connect to the bite trays or has to reconstruct the two trays building the anchors further away from the teeth. In addition, because the majority of bite trays are constructed of acrylic, it is very difficult to make adjustments.

Another limitation is the current methods of manufacturing the intraoral orthosis devices are extremely costly and time consuming. Currently, to manufacture an intraoral orthosis devices requires extensive hand work by a skilled technician. The technician will usually make several measurements of the bite trays and attempts to remove any excess material. If the technician removes too much material, the technician is required to add material and start the process over. The process of removing and adding material may take a skilled technician hours to create a single intraoral orthosis device. Due to the cost, many individuals whom would benefit from using an intraoral orthosis device cannot afford it.

Previous attempts to eliminate the need of a technician or dentist have failed. The Hilsen patent (U.S. Pat. No. 5,611,355) is an intraoral orthosis device and method that prevents snoring and sleep apnea. The device is made from a thermoplastic material that conforms to the user's mouth. In addition, the device includes a releasable fastening means for connecting the maxillary bite and mandibular bite. However, Hilsen still requires the manufacturing of a cast of the user's teeth. Hilsen fails to address the correct angle and vertex of the temporomandibular joint. In addition, the Hilsen device does not allow for the attachment of an arm.

Although the prior art did attempt to minimize the described limitations, the prior art did not resolve the limitation adequately. There remains a need for an intraoral orthosis device that is comfortable to wear. In addition, the device needs to be easily manufactured to decrease the cost.

### SUMMARY OF THE INVENTION

An intraoral orthosis device that has a bite tray nested in an outer tray. The bite tray has a higher deformation temperature than the outer tray at an elevated temperature. The outer tray stays rigid while the bite tray becomes malleable at a low temperature. The outer tray maintains the shape of the bite tray. The thickness of the outer tray's base varies to create a vertex and angle that, when the bite tray is formed encompassed with the outer tray, it maintains a similar angle to the temporomandibular joint. After formation of the inner bite tray to the users teeth, the outer tray is discarded. The intraoral orthosis device has two bite trays, a maxillary bite tray and a mandibular bite tray. The bite trays are connected by arms, releasable fasteners, or a combination of the two.

Another aspect of the invention is an exoskeleton that is placed around the bite tray. The exoskeleton is rigid and provides additional support and structure for the bite tray.

Accordingly, another aspect of the present invention is to provide a method to manufacture an intraoral orthosis device without the need of utilizing the skills of a technician or dentist. The new method allows the manufacturer to easily and quickly create a maxillary bite tray and mandibular bite tray that conform to the user's teeth.

### DESCRIPTION OF THE DRAWINGS

The invention may take form in certain parts and arrangement of parts, and preferred embodiment of which will be described in detail in the specification and illustrated in the accompany drawing, which for a part hereof:

FIG. 1 shows an exploded front side view of a bite tray, an outer tray, and two spacers; the type of bite tray utilized in this figure would use a releasable fasteners to connect to another bite tray as shown in FIG. 8;

FIG. 2 shows a cross section of the bite tray, the spacers, and the outer tray, illustrates the bite tray nested in the outer tray; in addition shows the varying thickness of the outer tray's base to create an angle;

FIG. 3 shows an exploded rear view of the outer tray, a mandibular bite tray, and the spacers, the type of bite tray utilized in this figure would use a releasable fasteners to connect to another bite tray as shown in FIG. 8;

FIG. 4 shows an exploded front side view of the outer tray and a maxillary bite tray with the location of the anchors for a maxillary bite tray, the type of bite tray utilized in this figure would use a releasable fasteners or arms to connect to the mandibular bite tray as shown in FIG. 8 or 9;

FIG. 5 shows an exploded front side view of the outer tray and the mandibular bite tray showing the typical location of the anchors for the maxillary bite tray, the type of bite tray utilized in this figure would use a releasable fasteners or arms to connect to the maxillary bite tray as shown in FIG. 8 or 9;

FIG. 6 shows the bottom view of the bite tray;

FIG. 7 shows the bottom view of the bite tray with one releasable fasteners connected to the bite tray and the other releasable fasteners exploded from the bite tray;

FIG. 8 shows a side view illustrating the maxillary bite tray and the mandibular bite tray connected by releasable fasteners;

FIG. 9 shows a side view illustrating the maxillary bite tray and the mandibular bite tray connected by a telescoping arm;

FIG. 10 shows a top view of the maxillary bite tray and the mandibular bite tray with the telescoping arms exploded from the bite trays, the dash lines shows the location of the anchors imbedded in the maxillary bite tray and the mandibular bite tray;

FIG. 11 shows a top view of the maxillary bite tray and the mandibular bite tray with the telescoping arms attached to the bite trays, the dash lines shows the location of the anchors embedded in the maxillary bite tray and a mandibular bite tray;

FIG. 12 shows a top view of the anchor with multiple petals;

FIG. 13 shows an exploded prospective bottom view of an exoskeleton, the bite trays, the spacers, and two inserts; and shows a bottom view of the exoskeleton, the bite trays, the spacers, and two inserts all connected;

FIG. 14 shows an exploded prospective bottom view of the outer tray, bite tray, and the inserts;

FIG. 15 shows an prospective bottom view with the outer tray removed, one side, the inserts, the spacers, one releasable fasteners, are connected to the bite tray, the other side shows an exploded view of the spacer, the insert and the releasable fasteners;

FIG. 16 is a flow diagram illustrating the steps of manufacturing of an intraoral orthosis device.

### DETAILED DESCRIPTION OF THE PREFERRED EMBODIMENTS

The following discussion describes embodiments of the invention and several variations of these embodiments. This discussion should not be construed, however, as limiting the invention to these particular embodiments. Practitioners skilled in the art will recognize numerous other embodiments as well. It is not necessary that the device have all the features described below with regard to the specific embodiment of the invention shown in the figures.

In the following description of the invention, certain terminology is used for the purpose of reference only, and is not intended to be limiting. Terms such as “upper”, “lower”, “above”, and “below,” refer to directions in the drawings to which reference is made. Terms such as “inwards” and “outward” refer to directions towards and away from, respectively, the geometric center of the component described. Terms such as “side”, “top”, “bottom,” “horizontal,” and “vertical,” describe the orientation of portions of the component within a consistent but arbitrary frame of reference which is made clear by reference to the text and the associated drawings describing the component under discussion. Such terminology includes words specifically mentioned above, derivatives thereof, and words of similar import.

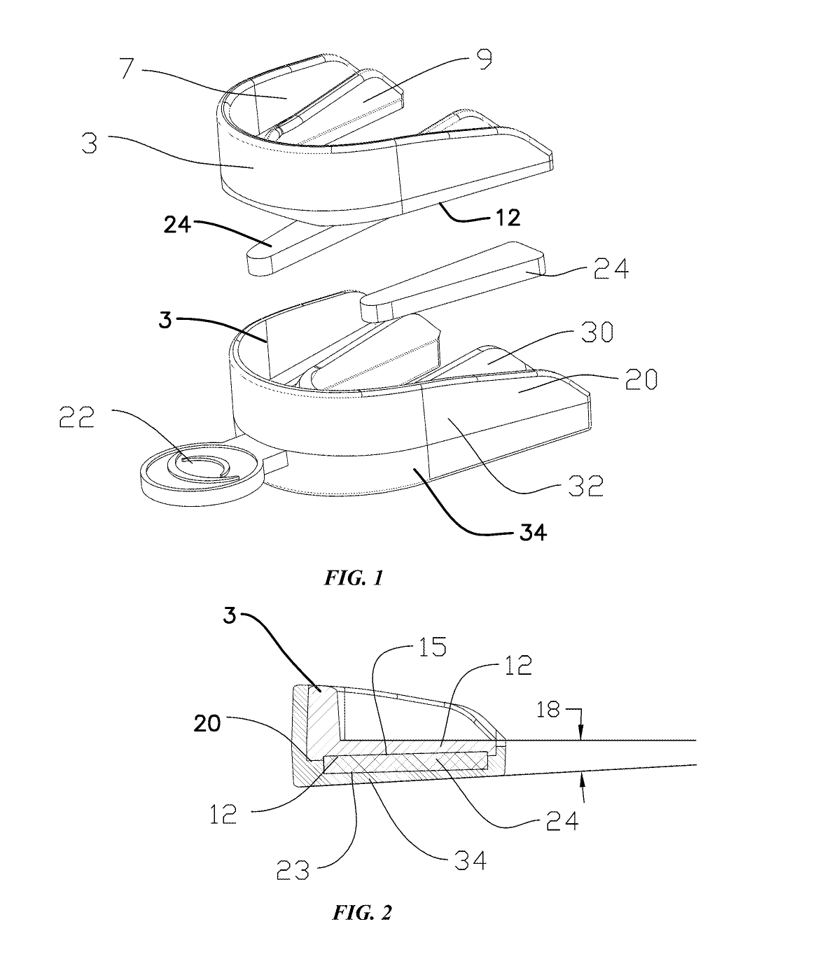

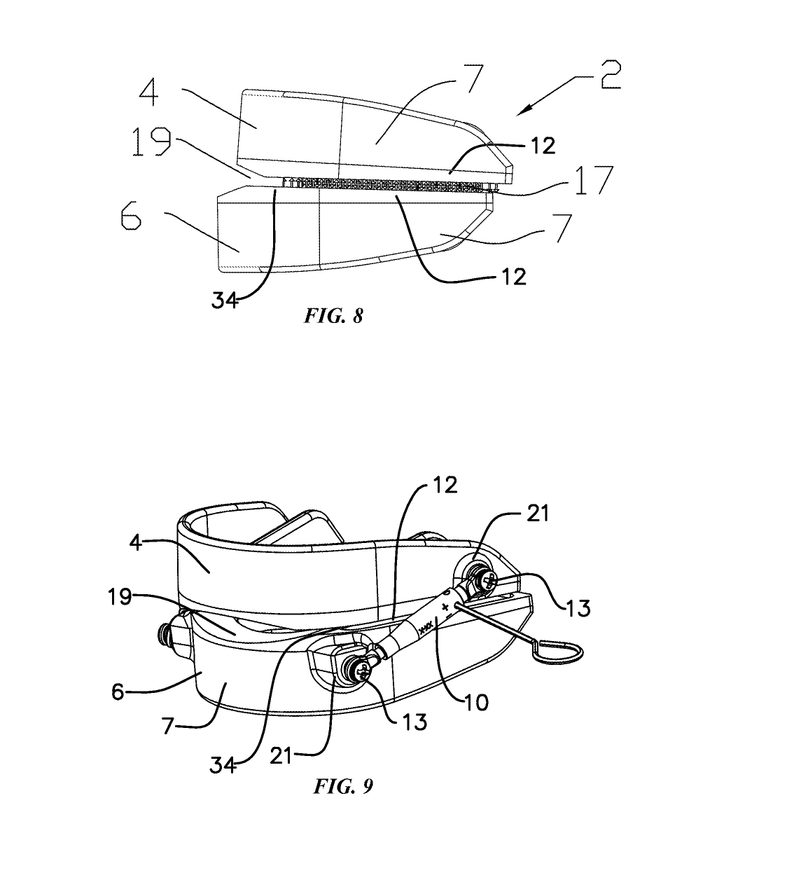

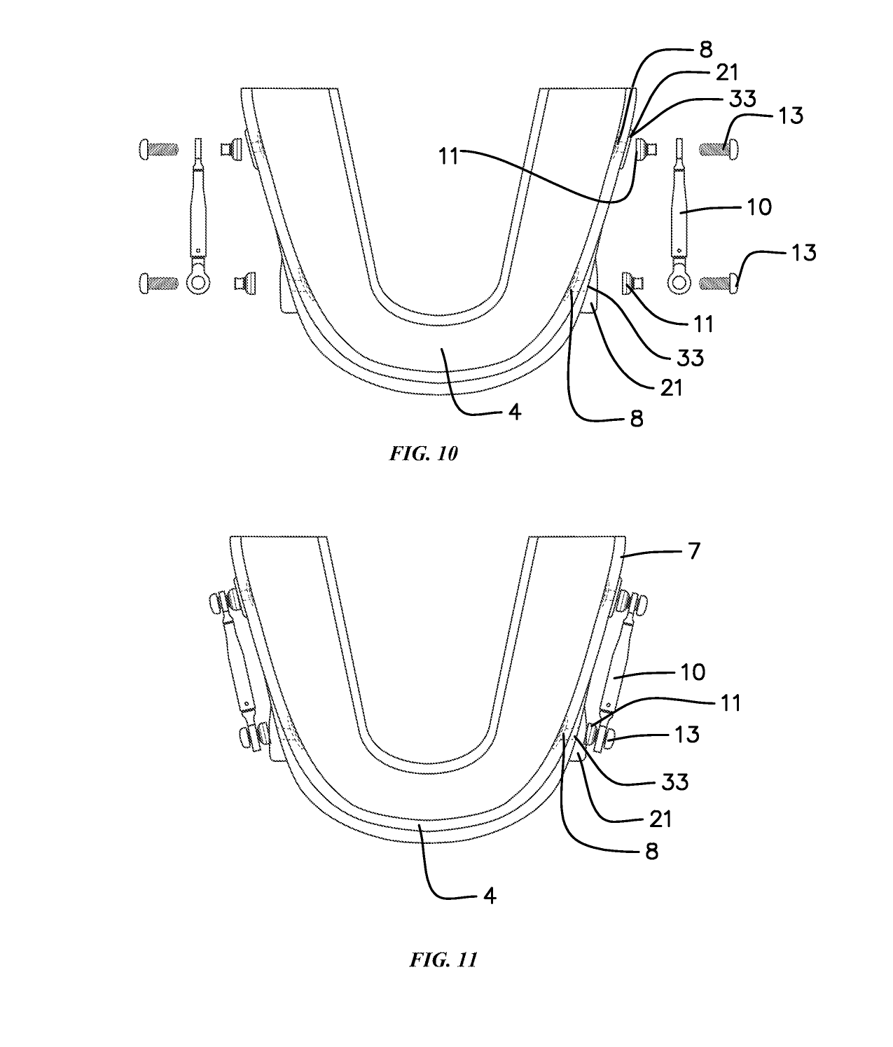

Referring generally to FIGS. 8 and 9, an intraoral orthosis device 2 embodying features of the present invention comprising two bite trays 3. As shown in FIGS. 1 and 2, the bite tray 3 has a U-shaped body sized for placement over a user's teeth. The bite tray 3 has an outer sidewall 7, an inner sidewall 9, and a crown 12. The outer sidewall 7 is located on the exterior circumference of the U-shape. The inner sidewall 9 is located on the interior circumference of the U-shape. The crown 12 is located on the base of the bite tray 3 from the back molar to the canine teeth.

As shown in FIGS. 8 and 9, the bite tray 3 consists of two types of trays: a maxillary bite tray 4 and a mandibular bite tray 6. FIG. 9 illustrates the maxillary bite tray 4 and the mandibular bite tray 6 connected by an arm 10. In practice, the most common type of arm 10 utilized is telescoping that allows the user to have some mobility of the jaw. FIG. 8 illustrates the maxillary bite tray 4 and the mandibular bite tray 6 connected by a releasable fastener 17.

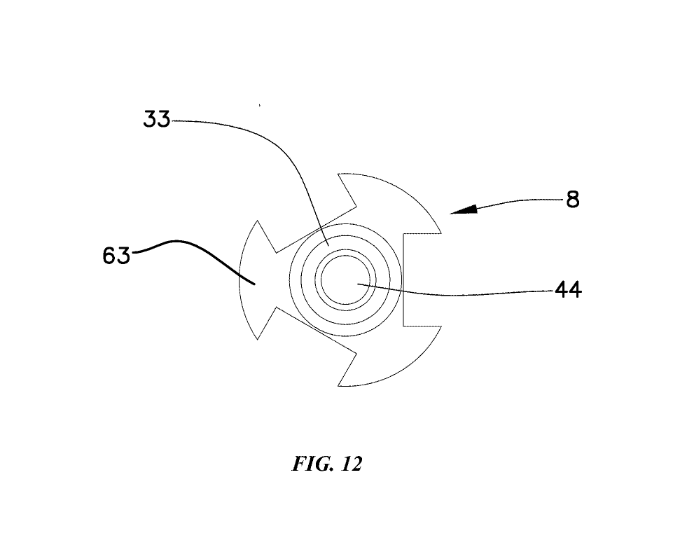

As shown in FIGS. 10 and 11, several anchors 8 may be imbedded in the outer sidewall 7 of the maxillary bite tray 4 and the mandibular bite tray 6. The interior of the anchor 8 has a threaded core 44 for receiving a screw 13. As shown in FIG. 12, the anchor 8 consists of several petals 63 extending from the longitude axis of the anchor 8. When the screw 13 is installed, a rotational force or torque is applied to the anchor 8. The petals 63 provide additional surface area for securing the anchor 8 to the bite tray 3 to resist the rotational force. In practice, three petals 63 are utilized, however, one skilled in the art will recognize that the size, length and number of petals 63 may vary. The anchor 8 has a tip 33 that is generally even with the outer surface of the bite tray 3. Preferably, the anchor 8 is manufactured from stainless steel, although any ridged material may be utilized. One skilled in the art will recognize there are several options for attaching the arm 10 to the bite tray 3. A washer 11 may be located between the arm 10 and the bite tray 3 to provide comfort to the user.

As shown in FIGS. 10 and 11, the anchor 8 is imbedded into the interior of the bite tray 3. To reinforce the anchor 8, a bulge 21 protrudes from the outer sidewall 7. The bulge 21 provides additional material around the anchor 8. The bulge 21 is generally the same material as the bite tray 3.

As described above, FIG. 8 illustrates the maxillary bite tray 4 and the mandibular bite tray 6 connected by a releasable fastener 17. The most common type of releasable fastener 17 is the trademarked Dual Lock that is made by 3M. However, other waterproof releasable connectors may be utilized. The user may utilize a combination of the arm 10 or the releasable fastener 17 to connect the maxillary bite tray 4 and the mandibular bite tray 6.

As shown in FIG. 9, a small concave opening 19 is situated on the front of the bite tray 3 of the crown 12. The concave opening 19 allows for the passage of air so the user can breathe more easily when the maxillary bite tray 4 and the mandibular bite tray 6 are in the closed position.

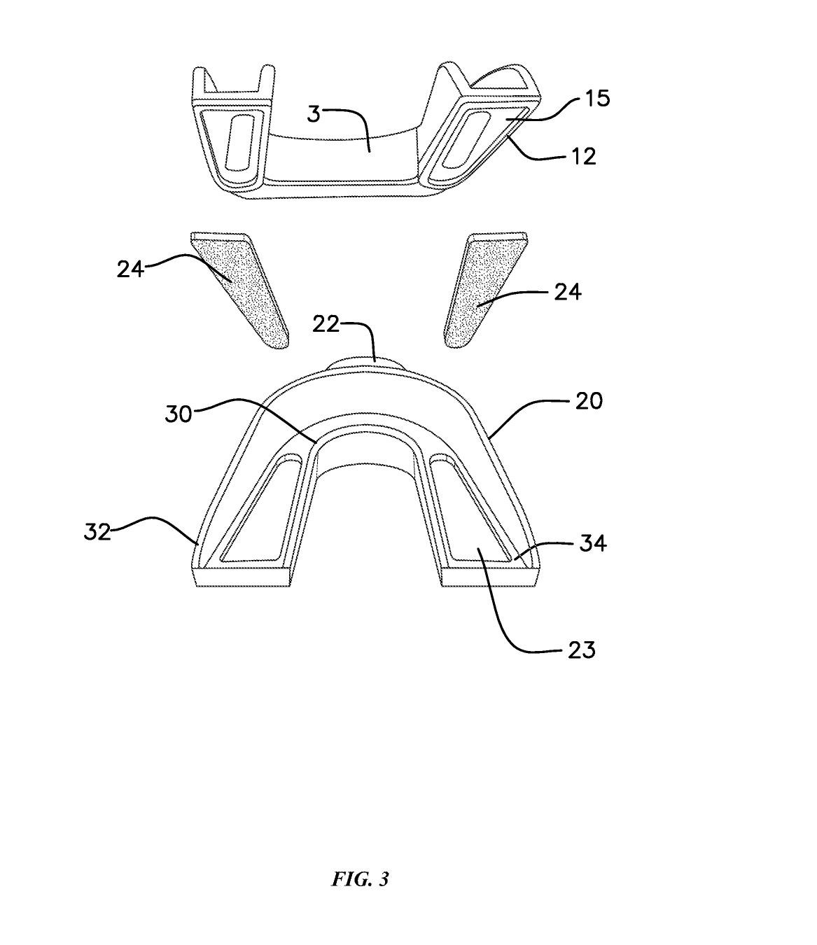

As shown in FIGS. 2 and 3, located on the crown 12, opposite of where the user's teeth will contact the bite tray 3, is a recess 15. The depth of the recess 15 is between 0.1 mm to 15.0 mm. The recess 15 is generally located on each side of the bite tray 3. As seen in FIG. 7, the recess 15 provides an area for the attachment of the releasable fastener 17. In addition, the recess 15 allows the intraoral orthosis device 2 to be less obstructive and bulky in the user's mouth.

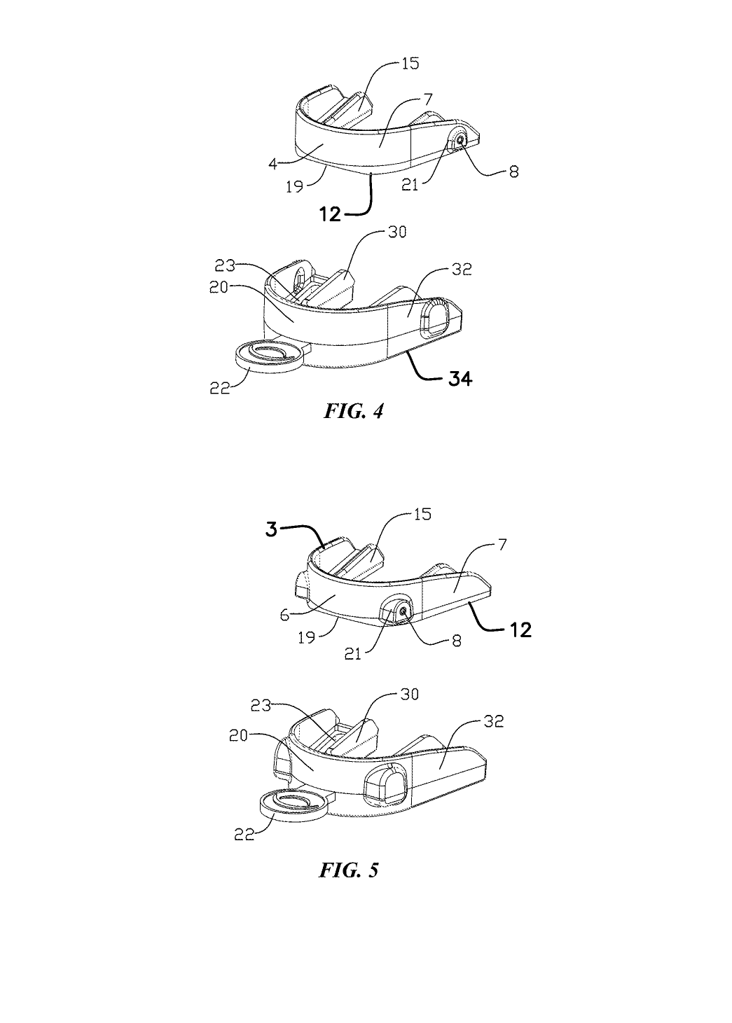

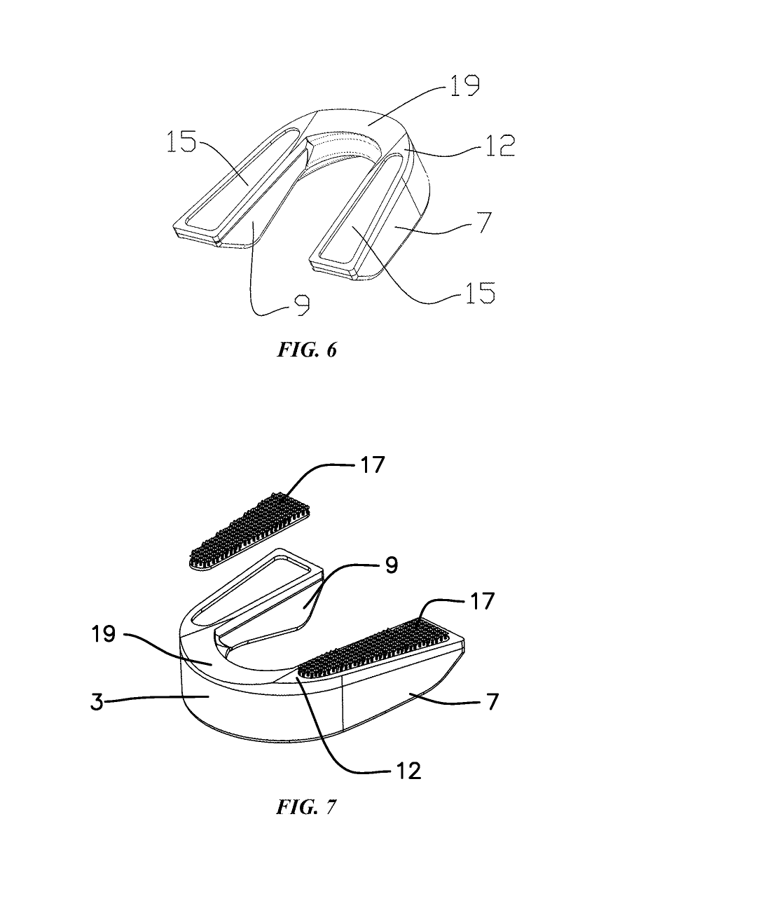

As shown in FIGS. 1 through 5, an outer tray 20 has the same U-shape body as the bite tray 3. The outer tray 20 shape is larger, such that the bite tray 3 may be nested inside the outer tray 20. The outer tray 20 is comprised of an exterior sidewall 32, interior sidewall 30, and a base 34. The thickness of the exterior sidewall 32 and interior sidewall 30 is such that it will support the bite tray 3 when the bite tray 3 is malleable and when the user's teeth apply pressure. Generally, the exterior sidewall 32 and interior sidewall 30 have the same thickness. The outline of the exterior sidewall 32 and interior sidewall 30 is such that it is comfortable when placed in the user's mouth for a short duration of time.

As shown if FIG. 2, the thickness of the base 34 varies from the front to the back of the outer tray 20. The front of the base 34 is greater than the thickness of the back of the base 34. The varying of the base's 34 thickness creates an angle 18. The angle 18 is similar to the angle and vertex of a temporomandibular joint. Achieving the proper angle 18 and vertex is essential for comfort to the user. As shown in FIG. 8, if the proper angle 18 is not achieved, the crown 12 of the mandibular bite tray 6 and maxillary bite tray 4 will not run parallel to each other and the releasable fastener 17 would not come in contact evenly resulting in the releasable fastener 17 not fastening. As illustrated in FIG. 9, when arms 10 are utilized it is also essential that the crown 12 of the mandibular bite tray 6 and maxillary bite tray 4 are parallel to each other.

As shown in FIG. 3, located on the base 34 is a cavity 23. The cavity 23 mirrors the location of the recess 15 when the bite tray 3 is nested in the outer tray 20. The depth of the cavity 23 depth is generally between 0.1 mm and 5.0 mm. The function of the cavity 23 is explained below.

As shown in FIG. 1 through 4, located at the front of the outer tray 20 is a grip 22. The grip 22 allows the user to easily handle the outer tray 20 and bite tray 3. The grip 22 becomes essential when the outer tray 20 and bite tray 3 are heated. Generally, the grip 22 is manufactured from the same material as the outer tray 20.

The bite tray 3 is comprised from a first material 35. The outer tray 20 is comprised from a second material 37. The first material 35 upon heating, often in boiling water, becomes soft and pliable. The second material 37 resists any deformation and has a low thermal conductivity such as silicone. When the bite tray 3 and the outer tray 20 are placed in heat, the outer tray 20 does not become as hot or soft. This allows the user to hold the outer tray 20, usually by the grip 22. In addition, the outer tray 20 provides support for the bite tray 3 when the bite tray is soft. The bite tray 3 and outer tray 20 are inserted into the user's mouth. The user applies pressure to the bite tray 3 and the bite tray 3 conforms to the user's teeth. As the bite tray 3 cools in the user's mouth, the bite tray 3 shapes permanently to the contour of the user's teeth.

As shown in FIGS. 2 and 3 to prevent the recess 15 from collapsing when the bite tray 3 is heated, a spacer 24 is placed in the recess 15 and cavity 23 of the outer tray 20. The spacer 24 is made of any ridged or semi-ridged material that has the same shape as the recess 15 and cavity 23. The spacer 24 assists in maintaining the correct position of the bite tray 3 when the bite tray 3 is nested in the outer tray 20. In addition, the spacer 24 assists in maintain the angle 18 and ensuring the crown 12 remains flat from the back molar to the canine on the bite tray 3. The spacer 24 is required to have a high deformation temperature to prevent distortion of the spacer 24 when heated. Upon completion of forming the bite tray 3, the outer tray 20 and spacer 24 is discarded or may be recycled.

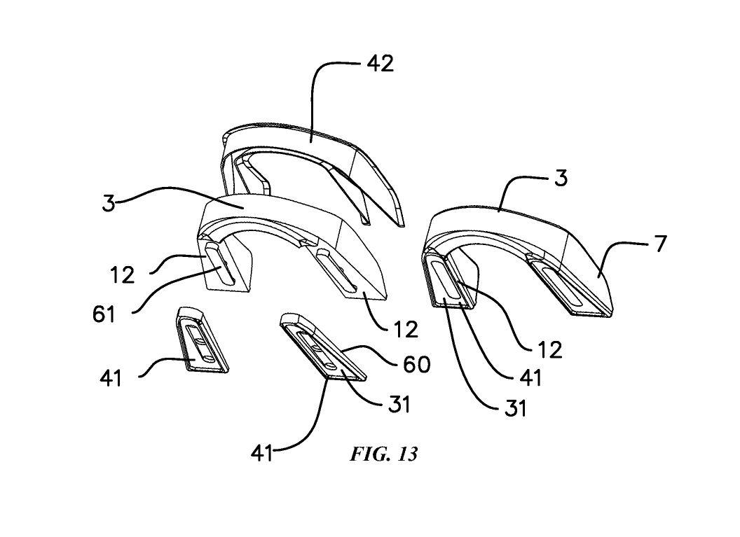

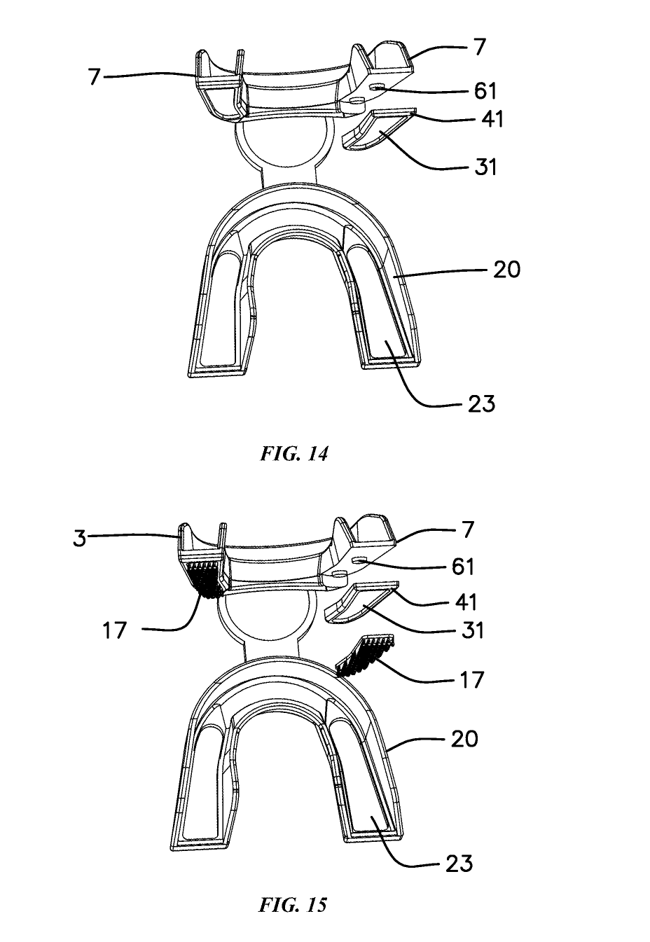

As shown in FIG. 13 through 15, another means to prevent the recess 15 from collapsing, maintaining the crown 12, and maintaining the angle 18 is an insert 41. The insert 41 is placed on the crown 12 of the bite tray 3. The insert 41 comprises an indent 31 and a receiver 60. When the insert 41 is utilized, the bite tray 3, has at least one pin 61 that interconnects to the receiver 60. The connection between the pin 61 and the receiver 60 ensures the proper alignment and placement of the insert 41. In practice, the shape, size, and number of the pin 61 and the receiver 60 may vary.

Located on the opposite side of the insert 41 from the receiver 60 is the indent 31. The indent 31 is similar in size and function as the recess 15. The indent 31 maintains the proper angle 18 and ensures the crown 12 is flat from the back molar to the canine. In addition, the insert 41 provides a surface for the releasable fastener 17. Furthermore, the indent 31 allows the intraoral orthosis device 2 to be less obstructive and bulky in the user's mouth. The depth of the indent 31 is between 0.1 mm to 5.0 mm.

The thickness of the insert 41 varies from the front to the back creating the angle 18 which is same as the angle and vertex of a temporomandibular joint. Another feature of the insert 41, is the overall thickness of the insert 41 may vary for the comfort of the user and to accommodate different size of users.

In addition to the features listed above, the insert 41 also allows for the bite tray 3 to be molded to the user's teeth with the releasable fastener 17 connect to the bite tray 3. As illustrated in FIG. 15, the insert 41 and releasable fastener 17 is attached to the bite tray 3. When the bite tray 3 is placed in the outer tray 20, the releasable fastener 17 is located in the cavity 23. The cavity 23 protects the releasable fastener 17 from damage when the user models the bite tray 3.

An additional feature of the invention is shown in FIG. 13, an exoskeleton 42 can be stationed over the bite tray 3. The exoskeleton 42, allows the user to utilize softer material for the bite tray 3. The exoskeleton 42 is comprised of a third material 78. The third material 78 is ridged and maintains it shape when heated or cooled.

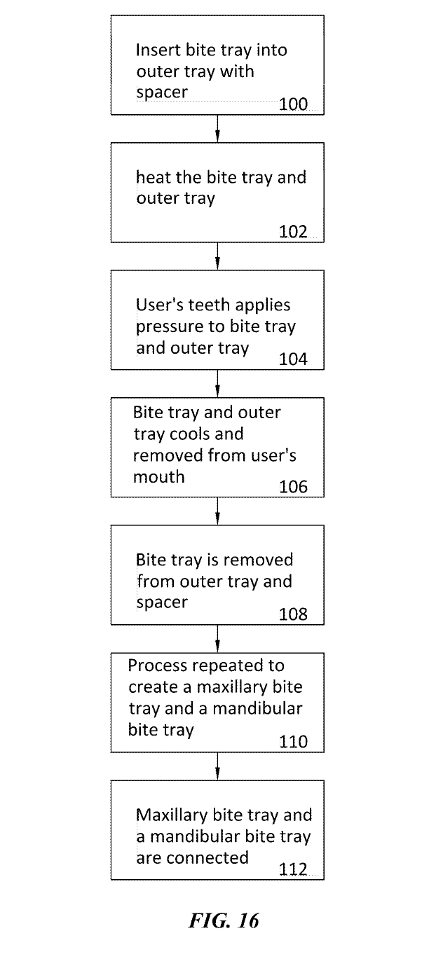

As Shown in FIG. 16, to manufacture the intraoral orthosis device 2, a user will follow the following steps. A spacer 24 is placed into the recess 15. The bite tray 3 is inserted into the outer tray 20 with the spacer 24 corresponding with the cavity 23 (step 100). The temperature of the now conjoined outer tray 20, spacer 24, and bite tray 3 is elevated (step 102). The most common heating method is heated water. When the bite tray 3 and outer tray 20 are sufficiently heated, the bite tray 3 and outer tray 20 are placed into the user's mouth. The user applies a pressure to the bite tray 3 and outer tray 20, causing the bite tray 3 to conform to the shape of the user's teeth (step 104).

When the bite tray 3 has cooled such that the deformation of the bite tray 3 has stopped, the bite tray 3 and outer tray 20 are removed from the user's mouth (step 106). The outer tray 20 and spacer 24 are removed and discarded (step 108). The bite tray 3 is trimmed and smoothed with a knife or heating element to remove any burrs or edges. The process is repeated to create the maxillary bite tray 4 and the mandibular bite tray 6 (step 110). As shown in FIGS. 8 and 9, the maxillary bite tray 4 and the mandibular bite 6 are connected by either the arms 10 or releasable fasteners 17 (step 112).

A variety of different permutations of the invention is contemplated, and not meant to be limited by this disclosure. The present invention is not limited to the preferred embodiments described in this section. The embodiments are merely exemplary, and one skilled in the art will recognize that many others are possible in accordance with this invention. Having now generally described the invention, the same will be more readily understood through references to the above descriptions and drawings, which are provided by way of illustration, and are not intended to be limiting of the present invention, unless so specified. Any element in a claim that does not explicitly state “means” for performing a specified function or “step” for performing a specified function, should not be interpreted as a “means” or “step” clause as specified in 35 U.S.C. §112.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings can be replaced by alternative features serving the same, equivalent, or similar purpose, unless expressly stated otherwise. Thus, unless expressly stated otherwise, each feature disclosed is one example only of a generic series of equivalent or similar features.

What is claimed:

1. An intraoral orthosis device comprising:
(a) a maxillary bite tray and a mandibular bite tray both having a U-shaped body sized for placement over a substantial portion of a user's teeth;
(b) an outer tray having a U-shaped body sized to embody the maxillary bite tray or the mandibular bite tray;
wherein, the outer tray having a base, the thickness of the base varies from the front of the outer tray to the back of the outer tray creating an angle;
wherein, the angle is similar to the vertex of a temporomandibular joint.

2. The device as recited in claim 1, wherein, said outer tray comprises a first material and said maxillary bite tray and said mandibular bite tray comprises a second material, wherein, the first material is rigid and resists any deformation, and the second material is soft and pliable at elevated temperatures.

3. The device as recited in claim 1, wherein said maxillary bite tray and said mandibular bite tray are connected by an arm.

4. The device as recited in claim 3, wherein said maxillary bite tray and said mandibular bite tray has at least one anchor.

5. The device as recited in claim 4, wherein said maxillary bite tray and said mandibular bite tray has a bulge, wherein the bulge provides support for said anchor.

6. The device as recited in claim 1, wherein said maxillary bite tray and said mandibular bite tray are connected by a releasable fastener.

7. The device as recited in claim 6, wherein said bite tray has a recess; wherein, said releasable fastener is located in the recess.

8. The device as recited in claim 6, wherein said outer tray has a cavity; wherein when said maxillary bite tray or said mandibular is nested in said outer tray, a spacer is located in the cavity and said recess;
wherein, the spacer provides support and maintains the position of said maxillary bite tray or said mandibular in said outer tray.

9. The device as recited in claim 2, an exoskeleton encases said maxillary bite tray or said mandibular bite tray;
wherein, the exoskeleton provides additional support for said second material.

10. The device as recited in claim 1, wherein said maxillary bite tray and said mandibular bite tray further comprises a crown.

11. The device as recited in claim 10, wherein an insert is connect to said crown, wherein, the insert maintains said angle.

12. The device as recited in claim 11, wherein said maxillary bite tray and said mandibular bite tray are connected by a releasable fastener that is attached to said crown.

13. The device as recited in claim 1, wherein said outer tray has a grip.

14. The device as recited in claim 1, wherein said maxillary bite tray and said mandibular bite tray has a concave opening.

15. A method for manufacturing an intraoral orthosis device comprising the following steps:
(a) nesting a bite tray having a U-shaped body sized for placement over a substantial portion of at least some of a user's teeth, within an outer tray having a U-shaped body sized for placement of the bite tray;
wherein, the outer tray having a base, the thickness of the base varies from the front of the outer tray to the back of the outer tray creating an angle;
wherein the angle has a similar vertex as a temporomandibular joint;
wherein the outer tray comprises a first material and the bite tray comprises a second material, the first material is soft and pliable at elevated temperatures; the second material resists any deformation;
(c) elevating the temperatures of the bite tray and the outer tray;
(d) placing the bite tray and the outer tray into the user's mouth;
(e) the user's teeth apply a pressure to the bite tray and outer tray causing the bite tray to conform to the shape of the user's teeth.

16. The method of claim 15 further comprising: repeating the process to create a maxillary bite tray and a mandibular bite tray.

17. The method of claim 15 further comprising: attaching an arm to said maxillary bite tray and said mandibular bite tray.

18. The method of claim 15 further comprising: attaching a releasable fastener to said maxillary bite tray and said mandibular bite tray.
